(19) United States
(12) Patent Application Publication
(10) Pub. No.: US 2005/0027312 A1
(43) Pub. Date: Feb. 3, 2005

(54) Balloon occlusion device

(76) Inventors: Andreas Hahn, Berg (DE); Marcus Gollner, Munchen (DE); Michael Wetzig, Munchen (DE); Peter Bernhardt, Munchen (DE)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/486,613

(22) PCT Filed: Aug. 9, 2002
(86) PCT No.: PCT/EP02/08967

(30) Foreign Application Priority Data
Aug. 10, 2001 (EP) 01118821.6

(51) Int. Cl. A61M 29/00

(52) U.S. Cl. 606/194

(57) ABSTRACT

The invention relates to a balloon occlusion device that comprises a cannula (1) and an occlusion device (3) to which a dialation liquid is fed. A core base (6) is disposed in the interior of the cannula (1) and reduces the volume in the interior of the cannula (1) and the mechanical flexibility of the cannula (1), thereby requiring less dilatation liquid and making the device easier to handle.

The invention relates to a balloon occlusion device in particular for use in cardiosurgery to occlude the aorta.

Balloon occlusion devices of this type are known and are used for cardiosurgical procedures in which the aorta has to be intraluminally blocked. The intraluminal occlusion of the aorta is carried out instead of clamping using a transversely attached clamp since in addition to damage to the vessel, this type of occlusion also carries the risk of relatively large particles detaching from the wall of the aorta and entering into the patient's bloodstream which can lead, inter alia, to cerebral embolisms and associated neurological deficits (cerebral infarct).

Balloon occlusion devices are known, for example, from EP 1 086 717 A1 and DE 195 15 933 A1.

From the point of view of the surgeon working with the balloon occlusion device, a simple and safe handling of the occlusion device is paramount, with particular importance being attached to handleability during insertion and positioning and to a quick occlusion. After the balloon occlusion device has been positioned and the aorta has been intraluminally occluded, the cannula has to maintain its position at a pressure of approximately 100 to max. 200 mmHg and a flow of 2 to 6 l/min. This means that comparatively high pressures must be generated for occlusion.

In view of the above, the problem to be solved by the invention consists of specifying a balloon occlusion device which is simpler to handle during insertion and positioning in the aorta and with which occlusion of the aorta can be carried out as quickly as possible without there being the risk of the balloon occlusion device moving out of position during the cardiosurgical procedure.

This problem is solved by a balloon occlusion device having the features of patent claim 1. Advantageous designs can be seen from the sub-claims.

The invention will be described in more detail below by means of an embodiment and with reference to the enclosed figures in which:

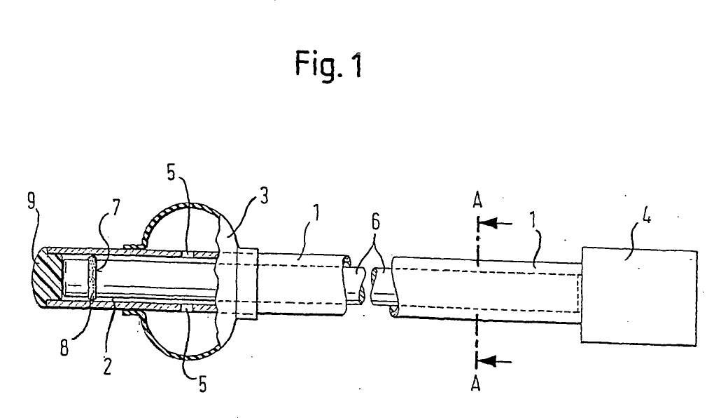

FIG. 1 shows a balloon occlusion device according to the invention;

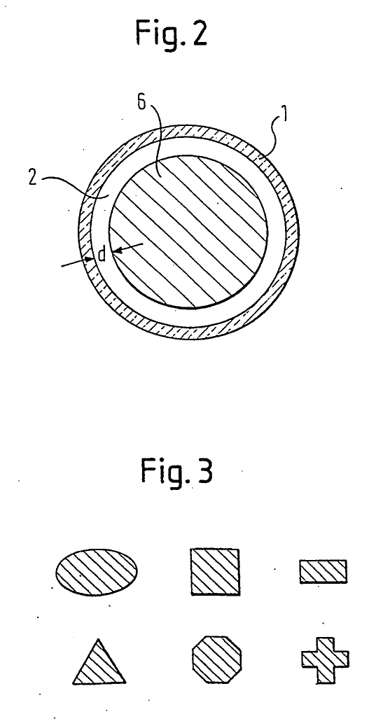

FIG. 2 shows a cross-section of the balloon occlusion device according to the invention at point A-A;

FIG. 3 shows several shapes of the cross-section of the core body of the balloon occlusion device according to the invention;

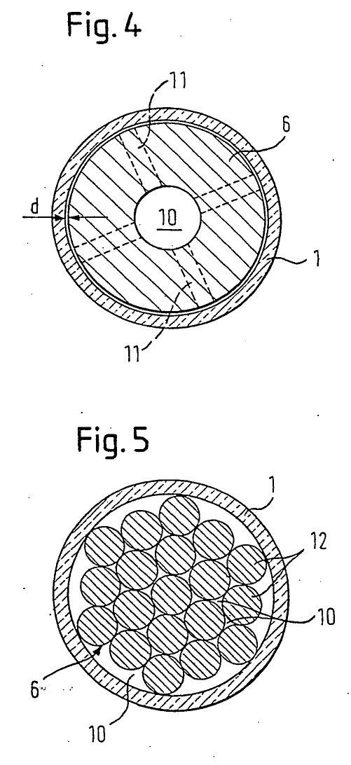

FIG. 4 shows a cross-section of a further design of the core body of the balloon occlusion device according to the invention;

FIG. 5 shows a cross-section of a further design of the core body of the balloon occlusion device according to the invention;

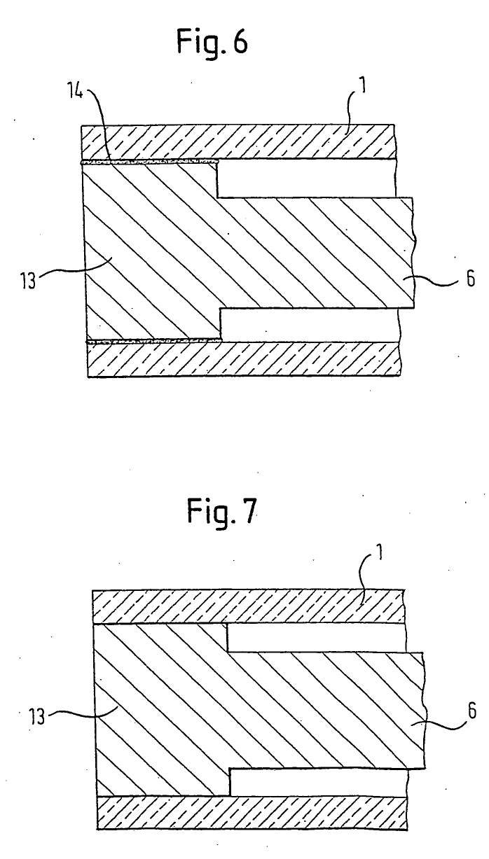

FIG. 6 shows a section view of a further design of the sealing means of the balloon occlusion device according to the invention;

FIG. 7 shows a section view of a further design of the sealing means of the balloon occlusion device according to the invention;

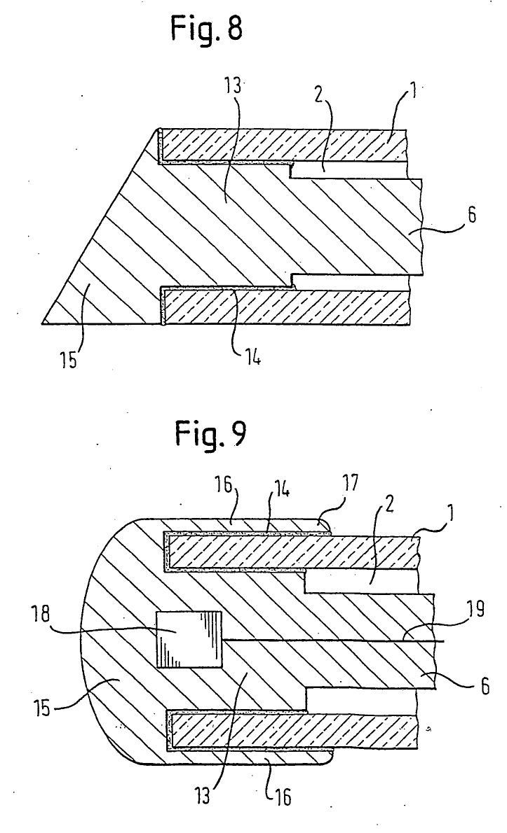

FIG. 8 shows a section view of a further design of the sealing means of the balloon occlusion device according to the invention;

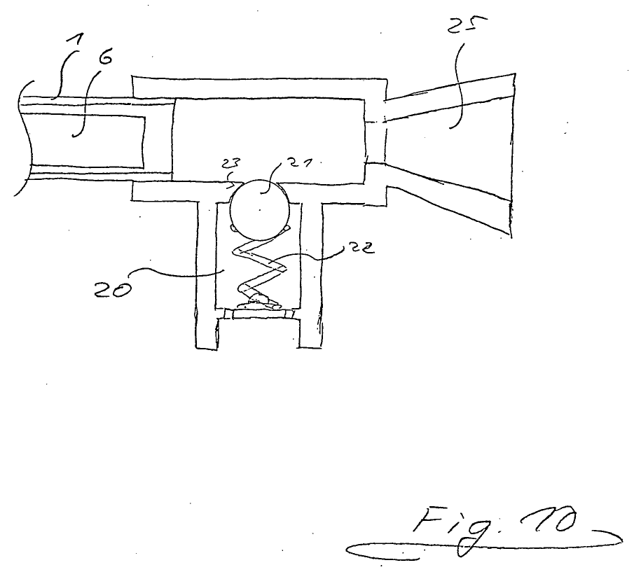

FIG. 9 shows a section view of a further design of the sealing means and sensors in the core body of the balloon occlusion device according to the invention; and

FIG. 10 shows a view of a safety valve on a balloon occlusion device according to the invention.

As shown in FIG. 1, the embodiment of the balloon occlusion device according to the invention described here consists of an elongated, flexible cannula 1 which inside surrounds a lumen 2 and at the distal end of which an occluding device, for example at least one occlusion balloon 3, is arranged, which is made of a flexible plastic, for example polyethylene, and which has a sufficient dimensional stability and rigidity to ensure a secure closure of the aorta following expansion and a secure position of the balloon occlusion cannula in the aorta. The diameter of the occlusion balloon 3 is adapted to the internal diameter of the aorta and is in the range of 20 to 30 mm.

In order to expand the occlusion balloon 3, it is supplied with a dilation liquid, for example physiological saline solution, via the cannula lumen 2 using a suitable supply means. For this purpose, a connection 4 is provided at the proximal end of the balloon occlusion device for the dilation liquid supply means which is, for example, a glass or plastic syringe. The connection is preferably designed according to the known “luer lock” principle which is characterised by the fact that by connecting the syringe and the connection 4, for example by insertion or screwing in, a pressure connection to the lumen 2 of the cannula 1 is created and that following removal of the syringe, the lumen 2 is sealed by the connection 4 such that pressure is maintained. The supplied dilation liquid flows out of the lumen 2 of the cannula 1 and into the occlusion balloon 3 via one or more through holes 5 and expands said occlusion balloon 3 according to the amount of dilation liquid supplied by the user.

According to the invention, a core body 6 is provided inside the cannula 1, which extends along the entire length of the cannula 1 in the embodiment shown here and partially occupies the cannula lumen 2 by taking up part, preferably 50%, of the cross-sectional area of the cannula 1 such that only the remaining part is available for supplying the dilation liquid to the occlusion device 3. As can be seen in FIG. 2, the core body 6 reduces the volume to be filled by the dilation liquid in the cannula lumen 2. This means that a much smaller amount of dilation liquid is sufficient to expand the occlusion balloon 3 since the cannula lumen 2 no longer has to be completely filled with dilation liquid. The remaining volume is determined by the gap d surrounding the core body 6 inside the cannula 1 along the entire longitudinal extent thereof. The presence of the core body 6 according to the invention inside the cannula 1 means that dilation of the occlusion device 3 can occur with a smaller amount of dilation liquid and thus quicker and occlusion of the aorta can thereby be achieved. The consequence of this for the surgeon is that he can achieve occlusion of the aorta and a fixing of the position of the balloon occlusion device with a very brief actuation of the supply means for the dilation liquid.

According to the invention, the core body 6 is simultaneously designed such that owing to its mechanical properties, it supports the surgeon when handling the balloon occlusion cannula. The shape, in particular the cross-section, and the material of the core body 6 are correspondingly selected such that the surgeon is supported when inserting the balloon occlusion device according to the invention, without insertion being hindered by too low a flexibility. This regularly leads to a reduction of the cannula lumen 2. This is achieved by means of a suitable selection of the material as well as by means of an adapted design of the shape and the cross-section, in particular the cross-sectional size. In the simplest case, the core body 6 is a solid rod of a flexible material that is suitable for medicinal applications, e.g. Teflon, which is arranged in the cannula lumen 2. The core body can comprise a reinforcement of a formable metal or a formable material, for example a suitable metal, e.g. nitinol, so that owing to the formability of the core body 6, the cannula 1 according to the invention can be brought into the desired shape before insertion, which the cannula then essentially maintains due to the formability of the core body 6. Deformation can be reversed at any time and reformation into the initial shape can be supported by the suitable selection of the material. The core body 6 preferably has a circular cross-section and is therefore adapted to the cross-section of the cannula 1. The core body 6 can also have the cross-sections shown in FIG. 3, i.e. elliptical, square, rectangular, triangular, polygonal or cross-shaped.

A further design of the core body 6 is shown in FIG. 4, in which the core body 6 has an external diameter which basically corresponds to the internal diameter of the cannula 1. So that dilation liquid can be supplied through the cannula 1, the core body 6 has a core body lumen 10 which extends from the proximal end to the area of the distal end of the core body 6 such that the dilation liquid supplied by the supply means can flow through the core body lumen 10. The gap d between the core body 6 and the cannula 1 is small and in borderline cases can even be avoided completely.

In the embodiment shown in FIG. 4, the core body lumen 10 has a circular cross-section, however other cross-sections can also be provided here. The core body 6 can furthermore have more than one core body lumen 10.

So that the dilation liquid can exit the core body lumen 10 and arrive in the occlusion balloon 3, the core body 6 comprises, at least in the region of its distal end, connecting openings 11 which are shown with dashed lines in FIG. 4 and which connect the core body lumen or lumens 10 with the surface of their core body 6 and thus with the cannula lumen 2 or directly with the through holes 5 of cannula 1. The dilation liquid then exits the core body lumen or lumens 10 and arrives in the cannula lumen 2 via the connecting openings 11 and then arrives from there or directly from the connecting openings 11 in the occlusion balloon 3 via the through holes 5. The number, the size and the cross-section of the connecting openings 11 can be freely selected within large ranges and are to be set according to the conditions of the cannula 1 and the occlusion balloon 3.

As shown in FIG. 5, the core body 6 can also be configured in the form of a bundle of individual or connected fibres 12. The fibre bundle 12 can thereby also have a total cross-section that basically corresponds to the internal diameter of the cannula 1, such that the clearances between the fibres 12 are to be considered as core body lumens 10, as is shown in FIG. 5. The fibre bundle 12 can, however, also be designed as core body 6 such that a gap d remains to the cannula, as is shown in FIG. 2 for a solid core body. The dilation liquid flows through the clearances 10 of the fibre bundle 12 and arrives in this manner in the cannula lumen 2 and in the occlusion balloon 3 via the through holes 5. In a further design, the core body can also be made of a porous material so that similarly to the fibre bundle, a core body having a plurality of core body lumens is provided.

As shown in FIG. 1, in a further design of the balloon occlusion device according to the invention, a fixing means is provided at the distal end of the core body 6, which fixes the core body 6 at the distal end in the cannula 1, i.e. which fixes the position of the core body 6 at the distal end. The core body 6 comprises a circumferential groove 7 for this purpose, in which a fixing ring 8 is received. The balloon occlusion device according to the invention can thereby be sealed so securely at the distal end by means of the core body 6 and the fixing ring 8 that a leakage of the dilation liquid at the distal end is certainly avoided. This means that the fixing means can additionally have a sealing function. Owing to the design, in particular the size and the material of the fixing ring 8 in coordination with the diameter of the core body 6 and the internal diameter of the cannula 1, not only a secure fixing but also a secure sealing of the balloon occlusion device at the distal end can be achieved, even at the pressures of up to approximately 0.5 bar in question here.

A sealing means 9 of the cannula lumen 2, is, however, also preferably provided at the distal end of the balloon occlusion device according to the invention, so that in addition to the fixing ring 7 on the core body 6, a further seal 9 prevents dilution liquid escaping from the cannula lumen 2. The sealing means can be the stopper 9 shown in FIG. 1.

As shown in FIGS. 6 to 9, the core body 6 can also be configured at the distal end as a sealing means 9.

In a first design according to FIG. 6, the diameter of the core body 6 is selected, at least in the area 13 of the distal end, such that the distal end of the core body 6 is adhesively connected with the cannula 1 by means of an adhesive layer 14, thereby sealing the cannula 1 at the distal end.

In a second design according to FIG. 7, an area 13 at the distal end of the core body 6 can be configured such that the outer surface of the core body 6 abuts the inner surface of the cannula 1 in this end area 13 and that a sealing of the distal end of the cannula 1 results.

In a further design according to FIG. 8, the distal end of the core body 6 can furthermore protrude from the cannula, the protruding section 15 having a diameter which is preferably greater than the internal diameter of the cannula 1. The protruding section 15 of the core body is preferably configured so as to support insertion of the cannula 1 according to the invention. For example, the protruding section 15 of the core body 6 has a rounded or, as shown in FIG. 8, a pointed shape, preferably pointed in a bevelled manner. FIG. 8 furthermore shows a design in which an adhesion area 13 on the core body 6 is provided at the distal end and the core body is adhered by means of adhesive 14 to the cannula 1 in this area 13 and in the area of the front edge of the cannula 1.

Finally, the protruding section 15 of the core body 6 can, as shown in FIG. 9, comprise a wall element 16 which extends from the distal end of the cannula 1 in the direction of the proximal end of the cannula 1, thereby forming a collar configured around the outside of the cannula 1 at the distal end thereof. The area 17 of the wall element 16 which faces the proximal end of the cannula is rounded so as not to impede withdrawal of the cannula 1 from the aorta. The collar 16 of the core body 6 preferably protrudes back to the area of the fixing ring 8. This prevents the cannula 1 expanding owing to internal pressure in the region of the ring 8 and thus the loss of the fixing function of the fixing ring 8.

One or more sensors, also ultrasound sensors or optical sensors, can be attached to the core body, as is indicated in FIG. 9 by reference number 18, said sensors detecting different physiological parameters such as pressure, temperature, oxygen partial pressure, etc. The core body 6 then serves as a carrier of these sensors and preferably also as a carrier of the sensor connector cables 19, via which the sensor signals are carried to the distal end and out of the cannula 1 according to the invention. The sensor or sensors 18 can be arranged on and/or in the core body 6 in the region of the cannula 1 and/or on the section 15 protruding from the cannula.

The core body 6 provided for a cannula according to the invention is also suitable for the application of markings, by means of which the user can establish how far the distal end of the cannula 1 has been inserted. For this purpose, the cannula 1 has to be made of a clear, or at least transparent, material.

In order to avoid damage occurring to the patient's vessel or to the occlusion balloon 3 owing to excessive pressure, a safety valve 20 is provided at the proximal end in an advantageous design of the balloon occlusion device according to the invention. This valve 20 is shown in FIG. 10, from which it can be seen that the safety valve opens if there is excessive pressure. If the doctor using the balloon occlusion device according to the invention plans the supply of the dilation liquid by means of a supply means arranged on the connection means 25 such that the limiting pressure of the safety valve 20 is exceeded, the ball 21 of the safety valve shown in FIG. 10 lifts against the restoring force of the spring 22 and allows dilation liquid to escape. The ball 21 of the safety valve 20 thereafter lowers back onto the valve seat 22 owing to the restoring force of the spring 22 so that the predetermined limiting pressure is maintained but is not, however, exceeded. This ensures that no damage occurs to the patient's vessel or to the occlusion balloon 3 if the safety valve is configured in this manner.

The example of a safely valve 20 shown in FIG. 10 for this advantageous design of the balloon occlusion device according to the invention comprises a ball 21 as a valve element which is forced into a valve seat 22 owing to the restoring force of a spring 20. Varying herefrom, other valve elements 21 with suitable resetting means 22 can be used in order to achieve the aim sought after with the design, i.e. to limit the pressure inside the balloon occlusion device.

1. A balloon occlusion device comprising:
a. a flexible cannula having through holes at a distal end thereof;
b. an occlusion device attached at the distal end of the cannula in an area of the through holes;
c. a connecting means for a supply means for supplying a dilation liquid which can be supplied via the cannula and the through holes into the inside of the occlusion device; and
d. a core body arranged inside the cannula, the core body being constructed and arranged on the one hand to reduce a volume inside the cannula and on the other hand to reduce mechanical flexibility of the cannula.

2. The balloon occlusion device according to claim 1, wherein the core body essentially extends along the entire length of the cannula.

3. The balloon occlusion device according to claim 1, wherein the core body has a circular, rectangular, square, polygonal or cross-shaped cross-section.

4. The balloon occlusion device according to claim 1, wherein the core body occupies at least 50% of the cross-sectional area of the cannula.

5. The balloon occlusion device according to claim 1, wherein the core body is fixed at the distal end of the cannula.

6. The balloon occlusion device according to claim 1, further comprising a fixing means that is provided at a distal end of the core body.

7. The balloon occlusion device according to claim 6, wherein the fixing means is configured as a fixing ring and wherein the core body includes a groove in which the fixing ring is arranged.

8. The balloon occlusion device according to claim 6, wherein the fixing means seals the cannula at the distal end of the cannula.

9. The balloon occlusion device according to claim 1, wherein the core body includes at least one core body lumen.

10. The balloon occlusion device according to claim 9, wherein the core body has connecting openings between the core body lumen and at least one of the cannula lumen and the through holes of the cannula.

11. The balloon occlusion device according to claim 1, wherein the core body is configured so as to be solid.

12. The balloon occlusion device according to claim 1, wherein the core body is configured as a fibre bundle.

13. The balloon occlusion device according to claim 1, wherein the core body is made of a porous material.

14. The balloon occlusion device according to claim 1, wherein the core body is made of a formable material, which can resume its initial shape.

15. The balloon occlusion device according to claim 1, further comprising a sealing means that is provided at the distal end of the cannula.

16. The balloon occlusion device according to claim 15, wherein the sealing means includes a stopper arranged in the distal end of the cannula.

17. The balloon occlusion device according to claim 15, wherein the core body includes a sealing area at a distal end thereof.

18. The balloon occlusion device according to claim 17, wherein the sealing area of the core body adheres to the cannula.

19. The balloon occlusion device according to claim 17, wherein the core body includes a section which protrudes from the distal end of the cannula.

20. The balloon occlusion device according to claim 19, wherein the section of the core body is pointed in a bevelled manner or rounded.

21. The balloon occlusion device according to claim 19, wherein the core body includes a wall element on the section, the wall element extending on the outside of the cannula from the distal end thereof in the direction of the proximal end of the cannula.

22. The balloon occlusion device according to claim 21, wherein the wall element of the core body is constructed and arranged to surround the core body.

23. The balloon occlusion device according to claim 21, wherein the wall element of the core body includes a rounded area facing the proximal end.

24. The balloon occlusion device according to claim 21, further comprising a fixing ring provided at the distal end of the core body, wherein the wall element extends into an area proximate the fixing ring.

25. The balloon occlusion device according to claim 1, wherein the core body includes markings configured and arranged to facilitate positioning of the cannula.

26. The balloon occlusion device according to claim 1, further comprising at least one sensor means for physiological parameters, the at least one sensor means being provided in or on the core body.

27. The balloon occlusion device according to claim 25, further comprising connector cables for the at least one sensor means, the connector cables being provided in or on the core body.

28. The balloon occlusion device according to claim 1, wherein the occlusion device includes a flexible balloon.

29. The balloon occlusion device according to claim 1, further comprising a safety valve that is provided at a proximal end of the cannula, the safety valve being constructed and arranged to prevent pressure inside the cannula from increasing to above a predetermined limiting pressure.
